(19) United States
(12) Patent Application Publication
(10) Pub. No.: US 2025/0059133 A1
(43) Pub. Date: Feb. 20, 2025

(54) PROCESS FOR RECOVERING AN ACETONITRILE

(71) Applicant: DE NEEF CHEMICAL PROCESSING N.V., Heist-op-den-Berg (BE)

(72) Inventor: Geert VERMEULEN, Heist-op-den-Berg (BE)

(21) Appl. No.: 18/721,778

(22) PCT Filed: Dec. 21, 2022
(86) PCT No.: PCT/EP2022/087363

§ 371 Date: Jun. 19, 2024

(30) Foreign Application Priority Data
Dec. 22, 2021 (BE) 2021/6037

(51) Int. Cl. C07C 253/34

(52) U.S. Cl. CPC C07C 253/34

(57) ABSTRACT

The invention concerns a process for recovering acetonitrile from an acetonitrile stream consisting of acetonitrile, 16 to 90 weight percent water, low boiling impurities having a boiling point below the boiling point of acetonitrile/water azeotrope, and high boiling impurities having a boiling point above the boiling point of acetonitrile, wherein the process successively comprises the steps of: A) introducing the acetonitrile stream into a first distillation column and. by performing a subatmospheric pressure distillation. separating a first acetonitrile/water azeotrope and the low-boiling impurities from the high-boiling impurities, wherein 1) the high-boiling impurities are substantially withdrawn through the bottom of the first distillation column: 2) the low boiling impurities are essentially withdrawn, as vapor, through the top of the first distillation column and 3) the first acetonitrile/water azeotrope and any remaining low-boiling impurities are withdrawn via a side outlet of the first distillation column: B) introducing the first acetonitrile/water azeotrope withdrawn through a side outlet of the first distillation column into a second distillation column, and by conducting a distillation at atmospheric pressure or higher pressure, separating a second acetonitrile/water-azeotrope of purified acetonitrile wherein the second azeotrope is withdrawn, as vapor, from the top of the second distillation column and acetonitrile is withdrawn from the bottom of the second distillation column, characterized in that a liquid alcohol having a boiling point in the range of 170-220° C. has been added to the acetonitrile stream.

### FIELD OF TECHNOLOGY

Acetonitrile is an organic compound, which is often used as a solvent.

### BACKGROUND

Acetonitrile is used as a solvent in various processes. Many low-value acetonitrile waste streams are disposed of by incineration. This is undesirable. Processes for recovering acetonitrile from low purity waste streams are known in the art. For example, WO2005044783discloses an improved process for recovering acetonitrile from a dilute aqueous low-grade acetonitrile stream. FIG. 2 thereof discusses a process in which use is made of so-called azeotropic distillation, also referred to as pressure-swing distillation, by first distilling at reduced pressure and then distilling at atmospheric pressure.

As mentioned, acetonitrile is used as a solvent in various processes, including the synthesis of polypeptides. The latter results, for example, in a waste stream consisting of acetonitrile, water and peptide residue. The peptide moieties can cause foaming upon recovery of the acetonitrile. Therefore, even using the improved process of WO2005044783, using azeotropic distillation, too much acetonitrile may be entrained in the bottom product of the first distillation tower. That means loss of valuable acetonitrile. Moreover, the waste water is then no longer biologically processable and must be sent to incineration.

US2019031581 discloses a method for separating a mixture of materials A and B by extractive distillation, using an extraction medium having a higher affinity to B than to A, wherein a feed stream comprising A and B is conducted towards the extraction medium in a column, wherein an overhead fraction comprising A and also a liquid fraction comprising B and extraction medium are obtained, the liquid fraction is collected on a collecting tray and heated and partially evaporated in a first indirect heat exchanger, the resultant vapor is released into the column and a non-evaporated proportion of the liquid fraction is collected as sump fraction in the sump of the column, the sump fraction is successively heated in a second indirect heat exchanger and a third indirect heat exchanger and in part evaporated, wherein the resultant vapor is at least in part released into the column, the sump fraction is separated in a stripper into a fraction comprising B and an extraction medium fraction, the extraction medium fraction is used as heating medium for the second heat exchanger, wherein a partially cooled extraction medium fraction is obtained, and an external heating medium is used for the third heat exchanger, and the partially cooled extraction medium fraction is used as heating medium for the first heat exchanger. The materials A and B may be acetonitrile and water. In this process ethylene glycol may be used as extraction medium. This process employs a second column, but this only serves to regenerate the extraction medium. This process therefore differs significantly from the process of WO2005044783. It is therefore important to further improve the process of WO2005044783, without radical changes in the process and the conditions. In addition, it is essential that an adjustment does not affect the quality of the acetonitrile. For example, the recovered acetonitrile must still be suitable for the synthesis of polypeptides. The problem to be solved by the present invention may therefore be regarded as how to enhance the separation in the distillation column. This problem has surprisingly been solved.

### SUMMARY OF THE INVENTION

The present invention relates to a process according to claim 1.

### FIGURES

FIG. 1 is a schematic representation of a preferred embodiment of the present process.

### DETAILED DESCRIPTION OF THE INVENTION

The process corresponds to the process as described in WO2005044783.

Preferably, use is made of a liquid alcohol having a boiling point in the range of 180-200° C. This can also be a mixture of alcohols. Various alcohols can be used in the current process. For example, 2-nonanol or 1-octanol can be used. Preferably, 2-ethylhexanol (2-EH) is used. It is important that the liquid alcohol or mixture thereof is below detection levels in the purified acetonitrile. A common specification on 2-EH for purified acetonitrile is 5 ppm. In the process according to the present invention, the content of 2-EH remains below the detection level determined by means of GC of 1 ppm. The amount of liquid alcohol or mixture thereof is preferably in the range of 0.01-0.5%, more preferably 0.02-0.04%, based on the weight of the acetonitrile stream.

As described in WO2005044783, the pressure during the distillation in the first distillation column may be between 150 and 400 mbar, and more preferably between 200 and 220 mbar. In view of the current modification of the process, the pressure during the distillation in the second distillation column may even be increased from atmospheric to overpressure, for example to 4 bar a, whereby a higher pressure can lead to a reduction of the recycle flow.

In order not to lose valuable acetonitrile, the second acetonitrile/water azeotrope from the second distillation column is preferably recycled to the first distillation column.

For applications in which strict purity requirements are imposed on the acetonitrile, the acetonitrile withdrawn from the second distillation column may be additionally purified or concentrated, preferably by means of an additional distillation and/or with the aid of activated carbon.

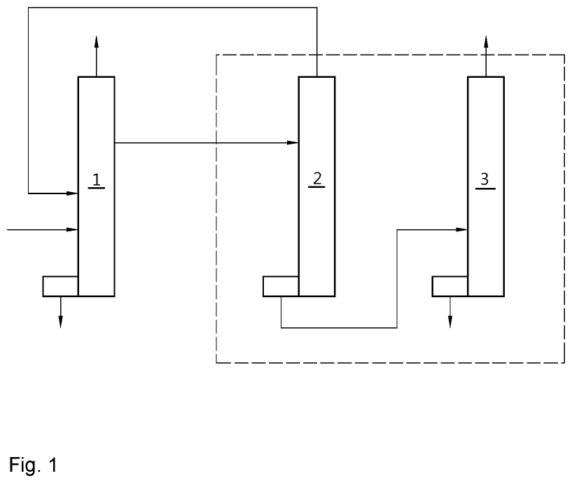

The preferred embodiment of the present invention is schematically shown in FIG. 1, wherein an acetonitrile stream is added to a distillation column 1. For example, the acetonitrile stream may contain 50% by weight of water. Distillation column 1 carries out a distillation at reduced pressure, preferably between 200-220 mbar. The low-boiling components are withdrawn from the top, and water and high-boiling components are withdrawn from the bottom. As mentioned, it is important that the waste water does not contain too much acetonitrile. The first azeotrope, withdrawn as a side stream, consisting mainly of acetonitrile with about 10% by weight of water, is added to distillation column 2. The second distillation column carries out a distillation at atmospheric pressure or higher, for example at 4 bar a. This results in a second azeotrope withdrawn from the top with about 24% by weight water, and an acetonitrile stream withdrawn from the bottom with less than 0.1% by weight water. The latter is preferably subjected to another distillation in order to separate pure acetonitrile at the top from high-boiling hydrophobic impurities and-if necessary-any salts at the bottom. This is shown schematically in distillation column 3. This second azeotrope is preferably recycled to the distillation column 2.

The invention can be illustrated by the following experiment performed with a solvent stream left over from a polypeptide production. In all cases, use was made of a stream containing approximately 20% by weight of acetonitrile, 1% by weight of high-boiling components and 1% by weight of low-boiling components. The stream contains peptide residues in varying amounts. The remainder of the solvent stream is water.

### Experiment 1

No problems with foaming were experienced in this stream. The waste stream from distillation column 1 therefore contains less than 0.1% by weight of acetonitrile. The amount of high-boiling ingredients has been increased to 1.25% by weight. The amount of low boiling components is below the detection level of 0.01%.

### Experiment 2

In a second stream, problems with foam formation were experienced by peptide residues. The waste stream from distillation column 1 therefore contains approximately 2% by weight of acetonitrile. This waste stream cannot therefore be processed biologically. In addition, about 10% of acetonitrile is lost as a result.

### Experiment 3

To the second stream with foaming peptide residues was added 2-EH. This surprisingly suppressed foaming. The waste stream from distillation column 1 therefore again contains less than 0.1% by weight of acetonitrile.

These experiments show that adding a liquid alcohol with a boiling point in the range 170-220° C. suppresses foaming by peptide residues and therefore leads to an improved process.

1. A process for recovering acetonitrile from an acetonitrile stream consisting of acetonitrile, 16 to 90 weight percent water, low boiling impurities having a boiling point below the boiling point of acetonitrile/water azeotrope, and high boiling impurities having a boiling point above the boiling point of acetonitrile, wherein the process successively comprises the steps of:
A) introducing the acetonitrile stream into a first distillation column and, by performing a subatmospheric pressure distillation, separating a first acetonitrile/water azeotrope and the low-boiling impurities from the high-boiling impurities, wherein
1) the high-boiling impurities are substantially withdrawn through the bottom of the first distillation column;
2) the low boiling impurities are essentially withdrawn, as vapor, through the top of the first distillation column; and
3) the first acetonitrile/water azeotrope and any remaining low-boiling impurities are withdrawn via a side outlet of the first distillation column;
B) introducing the first acetonitrile/water azeotrope withdrawn through a side outlet of the first distillation column into a second distillation column, and by conducting a distillation at atmospheric pressure or higher pressure, separating a second acetonitrile/water azeotrope of purified acetonitrile, wherein the second azeotrope is withdrawn, as vapor, from the top of the second distillation column and acetonitrile is withdrawn from the bottom of the second distillation column,
wherein a liquid alcohol having a boiling point in the range of 170-220° C. has been added to the acetonitrile stream.

2. The process according to claim 1, wherein the liquid alcohol has a boiling point in the range of 180-200° C.

3. The process according to claim 1, wherein the liquid alcohol is a mixture of alcohols.

4. The process according to claim 1, wherein the liquid alcohol is 2-ethylhexanol or a mixture thereof.

5. The process according to claim 1, wherein the amount of liquid alcohol or mixture thereof is in the range 0.01-0.5%, preferably 0.02-0.04%, calculated on the weight of the acetonitrile stream.

6. The process according to claim 1, wherein the pressure during the distillation in the first distillation column is between 150 and 400 mbar, and more preferably between 200 and 220 mbar.

7. The process according to claim 1, wherein the second acetonitrile/water azeotrope from the second distillation column is recycled to the first distillation column.

8. The process according to claim 1, wherein the acetonitrile withdrawn from the second distillation column is further purified or concentrated, preferably by means of an additional distillation.
